(19) United States
(12) Patent Application Publication
(10) Pub. No.: US 2026/0259478 A1
(43) Pub. Date: Sep. 3, 2026

(54) DEVICE FOR ASSISTING WITH THE CREATION OF IMAGE RECORDS, USE OF THE DEVICE AND METHOD FOR CREATING AN IMAGE RECORD

(71) Applicant: BAYER AKTIENGESELLSCHAFT, Leverkusen (DE)

(72) Inventors: Johannes LINZBACH, Leverkusen (DE); Sascha EILMUS, Leverkusen (DE); Ingo STEINMEISTER, Leverkusen (DE); Linlin ZHAO, Leverkusen (DE)

(21) Appl. No.: 18/870,887

(22) PCT Filed: Jun. 1, 2023
(86) PCT No.: PCT/EP2023/064669

§ 371 Date: Dec. 2, 2024

(30) Foreign Application Priority Data
Jun. 7, 2022 (EP) 22177629.7

(51) Int. Cl. G03B 15/05; G03B 17/56

(52) U.S. Cl. CPC G03B 15/05; G03B 17/561

(57) ABSTRACT

The subject of the present disclosure is a device for assisting with the generation of image recordings, and the use of the device for generating image recordings of plant parts and/or plant pests.

The subject of the present disclosure is a device for assisting with the generation of image recordings, and the use of the device for generating image recordings of plant parts and/or plant pests.

There are a large number of situations in which it can be expedient and/or useful to generate image recordings of plant parts and/or plant pests in order to analyze them in respect of various aspects.

J. G. A. Barbedo discloses, for example, a method for counting whiteflies on soybean leaves on the basis of image recordings of the leaves infested by the whiteflies (Using image recording processing for counting whiteflies on soybean leaves, Journal of Asia-Pacific Entomology, 2014, 17:685-694).

S. V. Mirnezami et al. disclose, for example, a method for automatically counting trichomes on the basis of image recordings of soybean leaves (Automated trichome counting in soybean using advanced image-processing techniques, Appl Plant Sci., 2020, 8(7): e11375).

J. Cho et al. disclose, for example, a method for automatically identifying whiteflies, aphids and thrips in greenhouses on the basis of image recordings of sticky traps in which the mentioned pests are located (Automatic identification of whiteflies, aphids and thrips in greenhouse based on image analysis, International Journal of Mathematics and Computers in Simulation, 2007, 1(1): 46-53).

Accordingly, there are a large number of tools for analyzing image recordings which allow a user (e.g. an agriculturalist) to determine the state of health of crop plants and/or to recognize and/or identify diseases and/or plant pests at an early stage. These tools require as the input one or more image recordings of a plant part and/or of plant pests. Such image recordings can be generated with digital cameras, which are nowadays built into almost every mobile telephone. However, for the automated analysis of the image recordings using methods of automated image analysis, it is necessary for the image recordings to have a certain quality. This means that the elements that are to be analyzed are imaged, for example, sharply and with high contrast. Furthermore, a predefined illumination of the object of which an image recording is to be generated and/or a predefined distance between the object and the image sensor of the camera are useful, since image recordings that are generated under predefined (always the same) conditions simplify automated image analysis. It is to be possible for the image recordings to be generated in the field and/or in the greenhouse; i.e. a portable device is required.

Proceeding from the described prior art, the technical object consisted in providing means for generating image recordings which are easy to handle, with which it is possible to generate image recordings that can be analyzed automatically using image analysis tools, which can be used in the field and/or in the greenhouse, and which can be used with a large number of different cameras. These means are further to permit the generation of image recordings under defined conditions (illumination, image size and/or the like).

This object is achieved by the subjects of the independent claims. Preferred embodiments are found in the dependent claims, the present description and the drawings.

The present invention firstly provides a device comprising
a body, wherein the body comprises an upper side and a lower side, wherein the body comprises a cutout between the upper side and the lower side,
a handle, which is connected to the body or is part of the body,
a mount for holding a camera on the upper side of the body above and/or inside the cutout,
a light hood, wherein the light hood comprises an inner side and an outer side, wherein the light hood comprises a first opening and a second opening opposite the first opening, wherein the light hood is attached to the lower side of the body, wherein the first opening of the light hood and the cutout of the body overlap at least partly,
one or more light sources, which emits/emit light onto the inner side of the light hood.

The present invention further provides the use of the mentioned device for generating image recordings of plant parts and/or of plant pests for the purpose of automatic image analysis.

The present invention further provides a method for generating an image recording, comprising:
providing a device comprising
   a body, wherein the body comprises an upper side and a lower side, wherein the body comprises a cutout between the upper side and the lower side,
   a handle, which is connected to the body or is part of the body,
   a mount for holding a camera on the upper side of the body above and/or inside the cutout,
   a light hood, wherein the light hood comprises an inner side and an outer side, wherein the light hood comprises a first opening and a second opening opposite the first opening, wherein the light hood is attached to the lower side of the body, wherein the first opening of the light hood and the cutout of the body overlap at least partly,
   one or more light sources, which emits/emit light onto the inner side of the light hood,
fastening a camera to the mount,
positioning the device relative to an object, wherein the light hood is positioned with the second opening on and/or over the object,
emitting light onto the inner side of the light hood,
generating an image recording of the object by means of the camera.

The present invention is elucidated in detail hereinafter without distinguishing between the subjects of the invention (device, use, method). The explanations that follow shall instead apply analogously to all subjects of the invention, regardless of the context (device, use, method) in which they are provided.

Where steps are stated in an order in the present description or in the claims, this does not necessarily mean that the invention is limited to the order stated. Instead, it is conceivable that the steps are also executed in a different order or else in parallel with one another, the exception being when one step builds on another step, thereby making it imperative that the step building on the previous step be executed next (which will however become clear in the individual case). The orders stated are thus preferred embodiments of the invention.

A subject of the present disclosure is a device.

The device has a body having a first side and a second side. The first side and the second side delimit the body in two directions to the outside (with respect to the surroundings/outside world).

The first side is referred to in this description also as the upper side; the second side is referred to in this description also as the lower side. When the device is being used by a user, the upper side usually faces the user, while the lower side faces away from the user. Preferably, the upper side and/or the lower side is/are flat (not curved). Preferably, the upper side and the lower side extend parallel to one another. Preferably, the upper side and the lower side are at a minimum distance from one another of more than 0.5 mm and/or at a maximum distance from one another of less than 10 cm.

The body has a cutout. The cutout is located between the upper side and the lower side; in other words, the cutout connects the upper side to the lower side; in other words, the lower side is reached from the upper side through the cutout.

The cutout can be designed, for example, as a tunnel or a notch. When seen in a plan view (e.g. in a view of the upper side and/or lower side of the body), the cutout can have an n-angular shape, wherein n is an integer greater than two (for example triangular, quadrangular (e.g. square or rectangular)), or a circular, oval, elliptical or other shape. Preferably, the cutout, when seen in a plan view, has a circular or rectangular shape. In the case of a cutout that is circular when seen in a plan view, the circle can have a diameter of, for example, from 3 mm to 50 mm. In the case of a cutout that is rectangular when seen in a plan view, the rectangle can have a side length of, for example, from 3 mm to 50 mm.

The cutout (i.e. the cut-out volume) can have, for example, the shape of a cylinder, a cuboid, a prism, a truncated cone or a truncated pyramid. Preferably, the cutout has a cylindrical shape or a cuboidal shape.

One or more optical elements can be mounted in the cutout. Examples of optical elements are lenses, stops, prisms, polarization filters, color filters, gray filters, diffractive optical elements, beam splitters and/or the like.

The device has a mount for a camera. Such a camera typically comprises an image sensor (camera sensor) and optical elements. The image sensor is a device for recording two-dimensional images from light by electrical means. It typically comprises semiconductor-based image sensors, for example CCD (CCD=charge-coupled device) or CMOS sensors (CMOS=complementary metal-oxide-semiconductor). The optical elements (lenses, stops and/or the like) serve for maximum sharpness of imaging of the object of which an image recording is to be generated on the image sensor.

In one embodiment, the camera is part of a mobile telephone, that is to say the mount for holding the camera is designed as a mount for holding a mobile telephone. The mount can, however, also hold a different camera.

The mount is preferably attached to the upper side of the body. The mount is designed such that the camera is attached to the upper side of the body above and/or inside the cutout. The mount ensures that the camera is brought into a defined position relative to the cutout. More specifically, the mount ensures that the image sensor of the camera is brought into a defined position relative to the cutout. More specifically, the mount ensures that the image sensor is positioned such that light passes through the cutout onto the image sensor. More specifically, the mount ensures that the image sensor is attached at a defined distance from the second opening of the light hood, such that light passes through the second opening of the light hood, through the first opening of the light hood and through the cutout onto the image sensor, and an object that is at a defined distance from the second opening of the light hood is imaged sharply on the image sensor.

The mount preferably has fastening means with which the camera can be fixed, such that the camera maintains its position in the case of a translational and/or rotational movement of the body. The fastening means can be designed as clamps, fastening straps, belts, screw connections and/or the like. Preferably, the fastening means allow the camera to be connected reversibly (releasably) to the body, that is to say the camera can repeatedly be connected to the body and the connection can repeatedly be released again.

If the camera is part of a mobile telephone, the mount can be designed, for example, as a case for the mobile telephone, into which the mobile telephone is embedded and by which the mobile telephone is held by a friction-based and/or interlocking connection.

The device has a light hood. The light hood comprises an inner side and an outer side. The light hood encloses a volume (also referred to below as the interior of the light hood). The light hood is delimited (to the inside) by the inner side of the volume. The outer side delimits the light hood with respect to the surroundings/outside world (to the outside). The light hood comprises a first opening and a second opening. The openings are preferably flat. The openings preferably extend parallel to and at a defined distance from one another. The distance can have, for example, a value in the range from 1 cm to 30 cm. Preferably, the distance is in a range from 3 cm to 20 cm.

When seen in a plan view (e.g. in the plane in which the first and/or second opening preferably extend), the first opening and/or the second opening can have an m-angular shape, wherein m is an integer greater than two (for example triangular, quadrangular (e.g. square or rectangular), pentagonal, hexagonal, heptagonal or octagonal), or a circular, oval, elliptical or other shape. Preferably, the first opening and/or the second opening, when seen in a plan view, has a circular or rectangular shape.

The light hood is attached to the lower side of the body such that the cutout of the body and the first opening of the light hood overlap at least partly. Preferably, the cutout and the first opening, when seen in a plan view, have the same shape. Preferably, the cutout and the first opening are in alignment with one another. Preferably, the cutout and the first opening are designed such that they are brought to congruence with one another, i.e. they overlap completely or almost completely, that is to say they are designed such that an observer perceives a single opening which leads from the interior of the light hood to the upper side of the body.

Preferably, the cutout of the body, the first opening of the light hood and the second opening of the light hood are arranged in alignment with one another.

Preferably, the second opening of the light hood has the same shape as the first opening of the light hood. Preferably, the second opening of the light hood is larger than the first opening of the light hood. The first opening of the light hood can, for example, be circular and have a diameter of from 5 mm to 50 mm, and the second opening of the light hood can, for example, likewise be circular and have a diameter of from 10 mm to 200 mm.

Preferably, the light hood has an axis of symmetry, wherein the axis of symmetry runs through the first and the second opening.

Preferably, the light hood (together with the volume that it encloses) has the shape of a spherical segment (e.g. the shape of a hemisphere), the shape of a truncated cone or the shape of a truncated pyramid. In the case of a light hood having the shape of a truncated cone or truncated pyramid, the first opening is preferably located in the top surface of the truncated cone or truncated pyramid, and the second opening is preferably located in the base surface of the truncated cone or truncated pyramid, and the lateral surface of the truncated cone forms, or the lateral surfaces of the truncated pyramid form, preferably the outer side of the light hood.

The device comprises one or more light sources. A light source is understood as being a source of electromagnetic radiation, wherein the wavelength of the electromagnetic radiation preferably lies at least partially in the range of the part of the electromagnetic spectrum that is visible to humans (380 nm to 690 nm). It is also conceivable that the light source emits electromagnetic radiation in the infrared range and/or in the ultraviolet range. The at least one light source can emit monochromatic or polychromatic light. Preferably, a user can choose between one or more wavelengths and/or one or more wavelength ranges of the emitted light. The light source can be, for example, a light-emitting diode (LED) or the light source can comprise a light-emitting diode.

The at least one light source is preferably mounted such that light is emitted onto the inner side of the light hood. The at least one light source is preferably mounted such that at least a portion of the emitted light is reflected from the inner side of the light hood in the direction toward the second opening of the light hood.

The at least one light source is preferably mounted such that emitted light is not emitted directly by the at least one light source through the first opening of the light hood and/or the second opening of the light hood. The at least one light source is preferably mounted such that emitted light is not emitted either directly through the first opening of the light hood or directly through the second opening of the light hood.

Preferably, the inner side of the light hood is designed such that it diffusely reflects (e.g. visible) light. Such diffuse reflection can be achieved, for example, by a white coating and/or rough surface. Preferably, the light hood is designed such that emitted light first strikes the inner side of the light hood and from there is transmitted directly or indirectly (i.e. after being reflected one or more times from other regions of the light hood) in the direction toward the second opening of the light hood and/or is not transmitted in the direction toward the first opening of the light hood. In other words: the at least one light source is preferably mounted such that it irradiates the inner side of the light hood, and radiation that is diffusely reflected from the inner side of the light hood passes through the second opening of the light hood onto the object of which an image recording is to be generated.

Preferably, the inner side of the light hood is provided with a coating that diffusely reflects the emitted light, while the outer side of the light hood is preferably provided with a coating that absorbs or reflects ambient light. The absorbing or reflecting coating ensures that no ambient light (e.g. sunlight) or as little ambient light as possible reaches the interior of the light hood, so as to avoid undesirable reflections in the image recordings.

Preferably, there are a plurality of light sources, which are mounted in the second opening of the light hood and/or above the second opening of the light hood (i.e. in the interior of the light hood) and are oriented such that they emit light onto the inner side of the light hood.

The device has a handle. The handle is attached to the body or the handle is part of the body. The handle assists a user in using the device. The user is able to pick up the device in one hand using the handle. The user can use his other hand, for example, to operate the camera and/or a mobile telephone, which can serve as the controller for the camera and/or the at least one light source.

Preferably, the device has one or more web elements which is/are laid across the second opening. Preferably, there are at least two web elements which run parallel to one another at a defined distance, for example at a distance of from 5 mm to 30 mm. It is also conceivable that web elements are arranged in the form of a square or rectangular or triangular or hexagonal grid. If the device is placed with the at least one web element on a plant part (for example a leaf), the at least one web element ensures that the plant part is leveled and/or oriented in parallel at a defined distance from the second opening. The at least one web element can be formed, for example, by a metal wire and/or a polymer thread. Preferably, the web element has a width of less than 1 mm, so as to cover as little of the surface of the plant part as possible when an image recording is being generated from an image of the plant part on the image sensor of the camera.

As an alternative to the at least one web element or in addition to the at least one web element, the device can comprise a support. The support can serve to place and/or fix a plant part on which the device is placed in order to generate an image recording of the plant part. The support is preferably flat and preferably has an extent that is greater than the second opening of the light hood. The support can comprise fastening means for fixing a plant part. The surface of the support that faces the second opening of the light hood can be of a defined color that ensures preferably maximum contrast with the plant part. The support can be provided with markings which can be used to calibrate the camera. With the aid of such markings, a size determination of an imaged object, for example, can be made possible.

The support can comprise fastening means with which the support can be fastened to the light hood and/or at a defined distance from the second opening of the light hood (wherein the distance may also be zero).

The device can comprise a source of electrical energy (e.g. an electrochemical cell, a battery, a solar cell, a fuel cell and/or the like) for supplying the electrically operated components (e.g. light source and/or camera and/or mobile telephone) with electrical energy. Preferably, the electrical energy for operating the camera and/or for operating the at least one light source and/or for operating a mobile telephone is provided by a battery, which can be part of the camera that is releasably connectable to the device and/or part of the mobile telephone or which can be a separate component. If the camera is part of a mobile telephone, for example, the battery of the mobile telephone can also be used to supply power to the at least one light source. For example, an electrical voltage can be tapped via a USB or lightning port of the mobile telephone and used to supply power to the at least one light source.

It is, however, also possible for there to be a separate battery (e.g. a so-called power bank) which supplies the camera, the at least one light source and/or a mobile telephone with electrical energy. It is possible for the device to have a camera and a mobile telephone, wherein the mobile telephone can be used to switch the at least one light source on and off and/or to operate the camera. The mobile telephone can also be used to determine location information. By means of a GPS receiver it is possible, for example, for the location at which one or more image recordings are generated using the camera to be determined and stored as information relating to the image recording or the plurality of image recordings. In an analogous manner, the time and/or the date at or on which one or more image recordings are generated can be acquired and/or stored using the mobile telephone, for example. Data acquired using the mobile telephone and/or image recordings can be transmitted by the mobile telephone via a mobile communications network to a separate computer system, for example, in order to analyze the image recordings on the separate computer system.

The device is portable, i.e. it can be carried by a user and used in the field and/or in a greenhouse to generate image recordings of plants, plant parts (e.g. leaves) and/or plant pests.

The subjects of the present disclosure are described in more detail below with reference to drawings, without any intention to restrict the subjects of the present disclosure to the features and combinations of features shown in the drawings.

In all of the drawings, the same reference signs have always been used for the various components of the device.

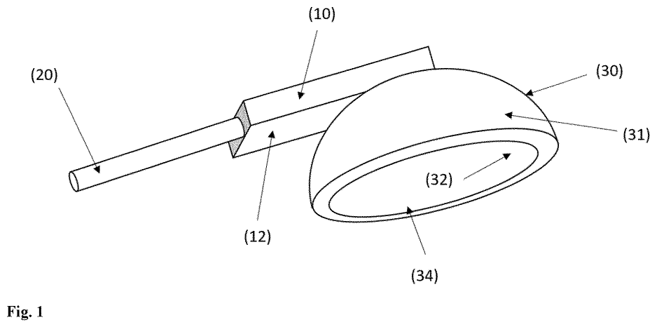

FIG. 1 schematically shows an embodiment of the device in a perspective illustration. FIG. 1 depicts the body (10). A handle (20) is attached to the body (20). A light hood (30) is attached to the lower side (12) of the body (10). The light hood (30) has the shape of a spherical segment. The light hood (30) comprises an outer side (31) and an inner side (32). The light hood (30) comprises a first opening (not visible in FIG. 1) and a second opening (34).

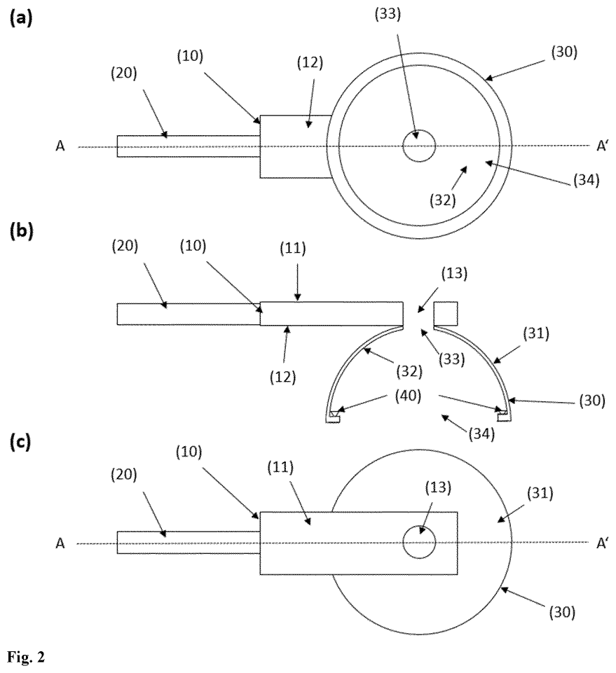

FIG. 2 (a), (b) and (c) show the same device as FIG. 1. FIG. 2 (a) shows the device in a bottom view (from the bottom). FIG. 2 (c) shows the device in a plan view (from the top). FIG. 2 (b) shows the device in a side view in cross section along line A-A′.

The device comprises a body (10). The body (10) comprises an upper side (11) and a lower side (12). The upper side (11) and the lower side (12) are flat and run parallel to one another.

The body (10) further comprises a cutout (13) between the upper side (11) and the lower side (12).

The device further comprises a handle (20). The handle (20) is connected to the body (10).

The device further comprises a light hood (30). The light hood (30) has the shape of a hemisphere. The light hood (30) is connected to the lower side (12) of the body (10). The light hood (20) comprises an inner side (32) and an outer side (31). The light hood (20) comprises a first opening (33) and a second opening (34) opposite the first opening (33). The second opening (34) is larger than the first opening (33). The first opening (33) and the second opening (34) have a circular shape. The first opening (33) is attached beneath the cutout (13) and is in alignment with the cutout (13).

Inside the light hood (30) there are mounted, above the second opening (34), light sources (40), which emit light onto the inner side (32) of the light hood (30). The light sources (40) are attached to a web element, which extends in the form of a circle around the second opening (34).

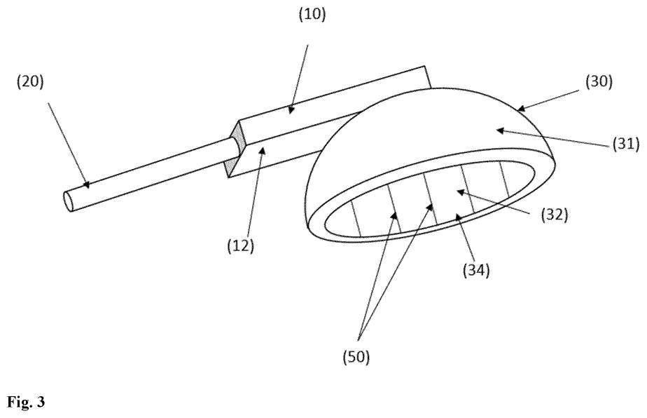

FIG. 3 schematically shows a further embodiment of the device in a perspective illustration. The device in FIG. 3 corresponds to the device shown in FIG. 1, with the difference that the device shown in FIG. 3 comprises web elements (50) which are laid across the second opening (34) of the light hood (30). The web elements (50) are arranged at a constant distance from one another; they run in parallel at the same distance from one another.

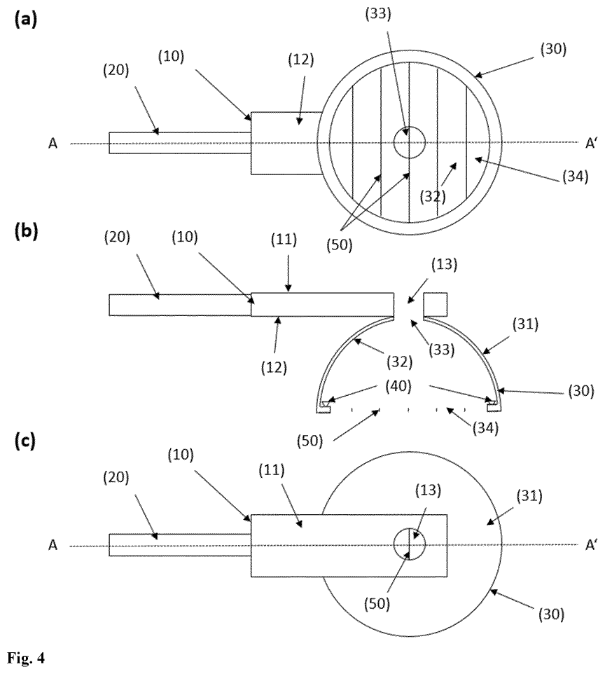

FIG. 4 (a), (b) and (c) show the same device as FIG. 3. FIG. 4 (a) shows the device in a bottom view (from the bottom). FIG. 4 (c) shows the device in a plan view (from the top). FIG. 4 (b) shows the device in a side view in cross section along line A-A′. The device in FIG. 4 (a), (b) and (c) corresponds to the device shown in FIG. 2 (a), (b) and (c), with the difference that the device shown in FIG. 4 (a), (b) and (c) comprises web elements (50) which are laid across the second opening (34) of the light hood (30). The web elements (50) are arranged at a constant distance from one another; they run in parallel at the same distance from one another.

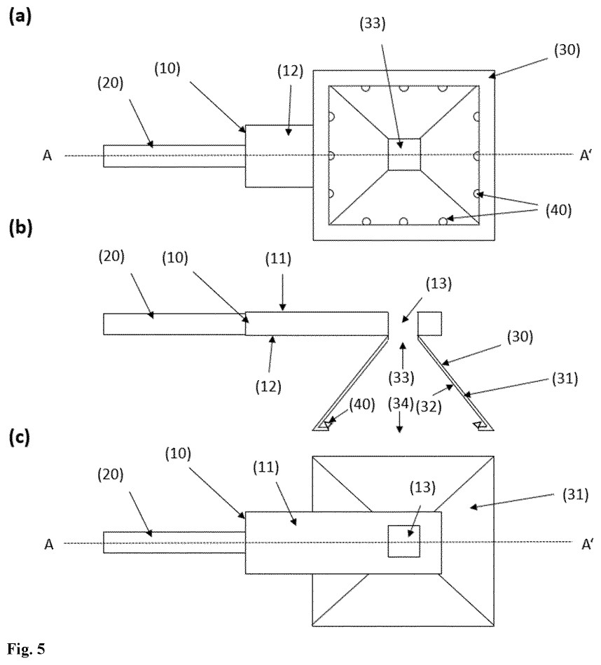

FIG. 5 (a), (b) and (c) schematically show a further embodiment of the device. FIG. 5 (a) shows the device in a bottom view (from the bottom). FIG. 5 (c) shows the device in a plan view (from the top). FIG. 5 (b) shows the device in a side view in cross section along line A-A′. The device in FIG. 5 (a), (b) and (c) corresponds to the device shown in FIG. 2 (a), (b) and (c), with the difference that the light hood in FIG. 5 (a), (b) and (c) has the shape of a truncated pyramid.

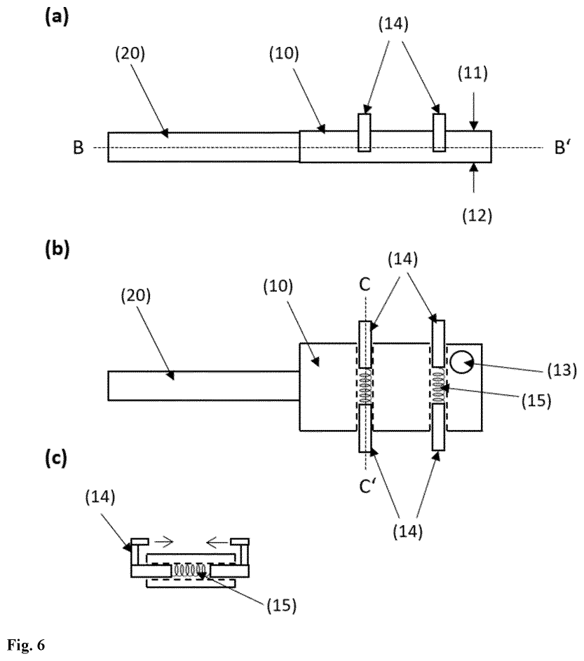

FIG. 6 (a), (b) and (c) show an embodiment of the device comprising a body (10), a handle (20) and a mount (14) for a camera. In the present example, the mount (14) is designed as a mount for a mobile telephone. FIG. 6 (a) shows the device in a side view, FIG. 6 (b) shows the device from the top in cross section along line B-B′. FIG. 6 (c) shows the device from the side remote from the handle, in cross section along line C-C′. The body (10) comprises an upper side (11), a lower side (12) and a cutout (13). The mount (14) comprises two pairs of elements which project from the body (10) on opposite sides of the body and are connected together inside the body by a tension spring. As a result of the tension springs, the mutually opposite elements (14) of the mount are acted upon by a force which moves the elements toward one another (indicated by the arrows in FIG. 6 (c)). The elements (14) of the mount have an angled shape, into which a mobile telephone can be clamped.

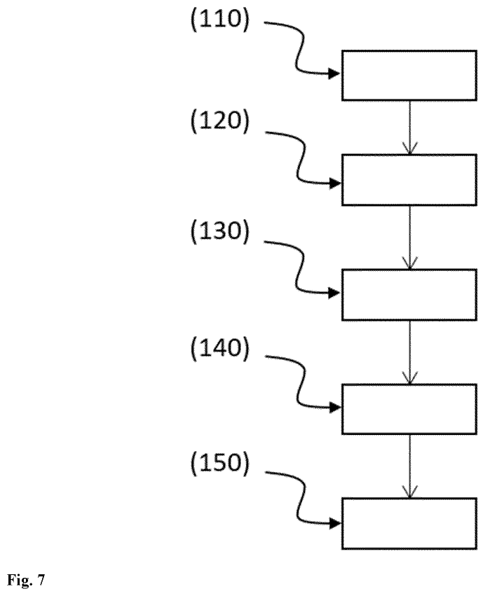

FIG. 7 shows an embodiment of the use of the device for generating image recordings of plant parts and/or plant pests, or a method for generating image recordings with the aid of the device, preferably in a field and/or in a greenhouse.

In a first step (110), the device is provided.

In a second step (120), a camera is fastened in the mount. Preferably, the camera is a camera built into a mobile telephone, i.e. the mobile telephone is fastened in the mount of the device. Here, the lens of the camera is arranged such that light from the object falls through the second opening of the light hood, through the first opening of the light hood and through the cutout in the body of the device onto the image sensor of the camera.

In a third step (130), a plant part is and/or one or more plant pests are placed in front of the second opening of the light hood (outside the light hood). It is, for example, conceivable that the device is placed with the second opening of the light hood at the front onto a plant part (e.g. a leaf) or onto a trap that contains one or more plant pests (e.g. a yellow board (yellow card) or a bowl trap). If the device comprises at least one web element, then the at least one web element can level the plant part or the yellow board. It is conceivable that a support is used, the plant part or the yellow board being placed on the support before the device is placed with the second opening of the light hood at the front onto the support and/or the plant part and/or the yellow board. It is conceivable that a plant part or a yellow board is clamped between the at least one web element and the support. It is conceivable that the support is connected to the light hood of the device.

In a fourth step (140), the light source is switched on, or the light sources are switched on if there is more than one. Preferably, the at least one light source emits electromagnetic radiation which strikes the inner side of the light hood, from there is reflected, preferably diffusely, in the direction toward the second opening of the light hood, then strikes the object of which an image recording is to be generated (e.g. plant part and/or one or more plant pests) and strikes the image sensor through the second opening of the light hood, through the first opening of the light hood and through the cutout of the body.

In a fifth step (150), an image recording is generated, i.e. the object imaged on the image sensor is acquired and a digital image recording is generated (for example a JPEG file or a BMP file or a TIFF file or an image recording in another format).

The image recording can be stored in a data memory, displayed on a screen, printed out by means of a printer and/or transmitted to a computer system. Preferably, the image recording is supplied to a computer program for automated image analysis.

1. A device comprising:
a body, wherein the body comprises an upper side and a lower side, wherein the body comprises a cutout between the upper side and the lower side;
a handle, which is connected to the body or is part of the body;
a mount for holding a camera on the upper side of the body above and/or inside the cutout;
a light hood, wherein the light hood comprises an inner side and an outer side, wherein the light hood comprises a first opening and a second opening opposite the first opening, wherein the light hood is attached to the lower side of the body, wherein the first opening of the light hood and the cutout of the body overlap at least partly; and
at least one light source configured to emit light onto the inner side of the light hood.

2. The device of claim 1, wherein the cutout has a cylindrical shape or cuboidal shape.

3. The device of claim 1, or wherein the mount is designed as a mount for a mobile telephone.

4. The device of claim 1, wherein the light hood has a shape of a circular segment, a truncated pyramid or a truncated cone.

5. The device of claim 1, wherein the first opening or the second opening has a circular shape or a rectangular shape.

6. The device of claim 1, wherein the inner side of the light hood is provided with a coating that diffusely reflects light.

7. The device of claim 1, wherein the outer side of the light hood is provided with a coating that reflects or absorbs light.

8. The device of claim 1, wherein the device comprises at least one web element which is laid across the second opening.

9. The device of claim 8, wherein the device comprises a plurality of web elements which are arranged at a defined distance from and parallel to one another.

10. The device of claim 1, wherein the device comprises a support which is or can be attached at a defined distance from the second opening.

11. A method for generating an image recording of a plant part or one or more plant pests, the method comprising using the device of claim 1 to generate the image recording.

12. The method of claim 11, further comprising:
providing the device;
fastening a camera to the mount;
arranging the plant part or one or more plant pests in front of the second opening of the light hood;
switching on the at least one light source; and
generating an image recording.

13. The method of claim 12, wherein the camera is part of a mobile telephone.

14. The method of claim 13, wherein the at least one light source is supplied with electrical energy by an energy source of the mobile telephone.

15. The method of claim 11, further comprising:
supplying the image recording to a computer program for automated image analysis.

16. (canceled)

17. The method of claim 14, further comprising supplying the image recording to a computer program for automated image analysis.
